(19) United States
(12) Patent Application Publication
(10) Pub. No.: US 2006/0035288 A1
(43) Pub. Date: Feb. 16, 2006

(54) Methods for detection of counterfeit liquids and foods

(76) Inventors: Lawrence R. Green, Tacoma, WA (US); Chris V. Rathe, Preston, WA (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(21) Appl. No.: 11/189,238

(22) Filed: Jul. 26, 2005

Related U.S. Application Data
(60) Provisional application No. 60/592,741, filed on Jul. 30, 2004.

(51) Int. Cl. G01N 33/53; G01N 33/00; G01N 33/20

(52) U.S. Cl. 435/7.5; 436/86; 436/84

(57) ABSTRACT

The present disclosure concerns methods of detecting counterfeit liquid and/or food products, particularly those used for human consumption. In certain embodiments, the claimed methods may comprise adding two or more markers to a liquid or food product, releasing the product into a stream of commerce, obtaining one or more samples purported to be the liquid or food product and assaying the samples for the presence of the markers, wherein the absence of at least one of the markers indicates that the product is counterfeit, fake or otherwise not authentic.

### FIELD

The present invention relates to methods for detecting counterfeit liquids and foods. In certain embodiments, the methods comprise adding two or more detectable marker substances to a liquid or food; distributing and/or selling the marked liquid or food; obtaining a sample of purported liquid or food; and assaying for the presence of the marker substances. In particular embodiments, the marker substance may be a vitamin or other liquid or food additive suitable for human consumption. In more particular embodiments, the liquid may be an expensive liquor.

### BACKGROUND

Counterfeiting of expensive liquid and/or food items is a major and growing problem. It has been estimated that the growth rate of counterfeit branded goods exceeds economic growth rates worldwide. (“The Growing Problem of Brand Counterfeiting and Diversion,” Digimarc White Paper, www.digimarc.com) Premium liquors are a common target of counterfeiting, either through refilling original bottles with an inexpensive substitute or by creating a look-alike product that mimics the packaging of the more expensive original. (“Consumers urged to be wary of counterfeit liquor,” Taipei Times, Nov. 29, 2003) In some markets, as much as fifty percent of apparent premium liquor products may be fakes. (Id.) In addition to the economic loss to the producers of premium beverage or food products, the counterfeit substitutes may contain harmful additives or contaminants, such as methanol in alcoholic beverages. (“Food agency warns about counterfeit liquor,” Media Release, NSW Food Authority, Jul. 23, 2004) Such counterfeit goods may contain sufficient amounts of contaminants to cause, for example, methanol poisoning in consumers. (Id.)

Product counterfeiting has been facilitated by the widespread availability and use of scanners, printing software and color printers, all of which may be run through desktop computers. The improvements in copying technology have led to recent upgrades in the security features of currency. However, the makers of branded products have been slower to adopt such measures. Further, many of the anti-counterfeiting techniques, such as inclusion of holographic or other hard to duplicate features in labels are circumvented by the practice of refilling original bottles with cheap substitutes.

Present approaches to counterfeit liquid detection focus on the packaging. For example, U.S. Pat. No. 6,549,131 discloses a method of marking objects by magnetically encodable regions, for example in a label. U.S. Pat. No. 6,613,715 discloses methods of using reversible, thermally sensitive labeling. Other approaches include the use of invisible optical watermarks and/or UV logos on packaging. (www.trustcopy.com) Such approaches may be of use to detect counterfeiting of the packaging, but can not detect the simple expedient of refilling an original package with an inferior substitute. A need exists in the field to be able to detect counterfeit liquid and/or food products that directly assays the contents of the packaging, rather than the packaging itself, to detect the presence of fake goods.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 is a flowchart illustrating an exemplary embodiment of a method for detecting counterfeit liquids and/or foods.

FIG. 2 illustrates an exemplary binary code label system that may be used in conjunction with marker substances.

FIG. 3 illustrates an exemplary embodiment of an analytical method for detecting markers in a liquid and/or food.

### DESCRIPTION OF ILLUSTRATIVE EMBODIMENTS

Additional details illustrating exemplary embodiments of the present invention are disclosed in U.S. patent application Ser. Nos. 09/974,089 (now issued U.S. Pat. No. 6,767,733); Ser. No. 10/425,222, filed Apr. 29, 2003; Ser. No. 10/373,546, filed Feb. 24, 2003; Ser. No. 10/373,408, filed Feb. 24, 2003; and U.S. Provisional Patent Application 60/540,720, filed Jan. 29, 2004, the text of each of which is incorporated herein by reference.

### DEFINITIONS

Terms that are not otherwise defined herein are used in accordance with their plain and ordinary meaning.

As used herein, “a” or “an” may mean one or more than one of an item.

As used herein, the terms “analyte” and “marker” mean any compound, molecule or aggregate of interest for labeling a liquid and/or food item. Non-limiting examples of markers include a protein, peptide, carbohydrate, sugar, polysaccharide, glycoprotein, lipid, antigen, antibody, substrate, metabolite, cofactor, nutrient, vitamin, or any other molecule or atom. In preferred embodiments, substances used as markers are those that may be added to a liquid or food product for human consumption, without requiring approval from the U.S. Food and Drug Administration (“FDA”), for example, a low concentration of a vitamin. In other preferred embodiments, the markers used are stable for extended periods, such as weeks or months, in the liquids and/or food products to be marked, such as liquors containing alcohol.

As used herein, “capture molecule” or “probe” refers to a molecule or aggregate that has binding affinity for one or more markers. Within the scope of the present invention virtually any molecule or aggregate that has a binding affinity for some marker of interest may be a “probe.” “Probes” include, but are not limited to, polyclonal antibodies, monoclonal antibodies, antibody fragments, FAb fragments, humanized antibodies, single-chain antibodies, chimeric antibodies, affibodies, oligonucleotides, polynucleotides, nucleic acids, aptamers, binding proteins, receptor proteins, biotin, streptavidin, avidin and any other known ligand that can bind to at least one marker molecule.

The terms “detect,” “detection” and “detecting” are used herein to refer to the use of an assay or procedure that is indicative of the presence of one or more markers in a sample. It will be appreciated by those of skill in the art that all assays exhibit a certain level of false positives and false negatives. However, an analytical method may be of use to detect a marker even where there is some level of false positives or false negatives. In preferred embodiments, the level of false positives and/or false negatives is less than ten percent, more preferably less than five percent.

Method of Detecting Counterfeit Liquids and/or Foods

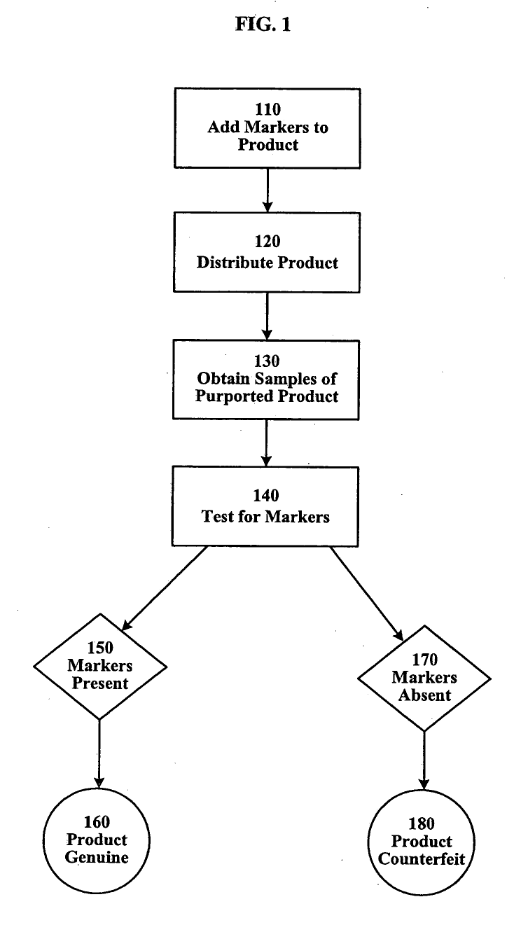

FIG. 1 illustrates an exemplary method of detecting counterfeit liquids and/or foods. The method is based on the addition of two or more marker substances to a liquid or food product, the introduction of the liquid or food product into a commercial distribution stream, obtaining samples of the purported liquid or food product from the commercial distribution stream, and testing the samples for the presence of the markers.

As illustrated in FIG. 1, in 110 two or more markers are added to a liquid or food product. In certain preferred embodiments, the product may be an expensive liquor. However, any type of liquor may be marked and validated, including but not limited to beer, wine, champagne, vermouth, vodka, scotch, whiskey, bourbon, brandy, cognac, gin, rum, tequila, schnapps, absinthe, akvavit, amaretto, amarula, arak, cassis, compari, Contreau, curacao, Drambuie, Galliano, Grand Marnier, Jagermeister, kahlua, kirsch, Midori, ouzo, pernod, sambuca, triple sec and sake. The skilled artisan will realize that the disclosed methods are not limited to liquors but may be applied to any type of consumable liquid or food product that is subject to counterfeiting.

After the markers are added to a liquid or food product, the product is released for distribution 120 into the stream of commerce. Samples of purported product may be obtained 130 anywhere in the distribution system, starting at the manufacturing facility and ending with the consumer. In non-limiting examples, samples may be obtained from a store or market, from a wholesale distributor or from a bar or restaurant.

Once a sample of the purported product is obtained, it is analyzed for the presence of the markers 140. The skilled artisan will realize that, depending on the marker used, a wide variety of analytical methods are suitable for detecting the presence or absence of the markers in the sample. Non-limiting examples include mass-spectroscopy, HPLC, capillary electrophoresis, thin-layer chromatography, ELISA, gel electrophoresis, column chromatography, immunoaffinity chromatography, NMR and lateral flow detection. For example, U.S. Pat. No. 5,506,109 discloses a method for detecting vitamin B12 by immunoassay. U.S. Pat. No. 6,426,194 discloses an enzymatic assay method for vitamin B6. HPLC methods are known for analysis of vitamins A and D. (“Report on the review of analytical method development under the food standards agency's research programme No. 8,” www.foodstandards.gov.uk). U.S. Pat. No. 5,558,870 discloses a colorimetric assay for vitamin C, based on reaction with the dye 2,6-dichlorophenolindophenl (DCIP). Any analytical method known in the art may be used to detect markers in samples.

In the simplest case, two markers are added to the original product prior to distribution. In preferred embodiments, the analytic method used tests for the presence or absence of the two markers. The determination of whether a marker is present or absent is a function of the sensitivity (detection limit) of the analytical method used and the assay readout is a binary yes/no. Either a given marker is present (detectable by the analytical method) or it is absent. If both markers are present 150, the sample is passed as authentic 160. The finding that one or both markers are absent 170 (not detectable by the analytical method) indicates that the sample is a counterfeit product 180. Additional steps may be taken if a counterfeit product is detected. For example, the product may be confiscated or additional samples obtained and subjected to further testing to confirm that the product is in fact counterfeit. Depending on the sensitivity and specificity of the analytical method, it is anticipated that some level, preferably less than 10%, more preferably less than 5%, most preferably less than 2%, of false positives and/or false negatives may result. Confirmation of the initial results may be performed to eliminate the possibility of a false positive result.

In other preferred embodiments, an analytical method may be performed in the field with an immediate yes/no readout obtained. Such methods may, for example, employ a “dip-stick” type of analysis using lateral flow technology, as illustrated in Example 1 below. In alternative embodiments, the analytical method may require more sophisticated equipment, such as an HPLC unit, that is not readily adapted for field use. In other alternatives, the sample may require processing prior to analysis. This may be true, for example, with solid foods that may need to be homogenized, ground up, filtered, extracted, centrifuged, dialyzed, separated and/or otherwise processed before analysis. In such cases, samples of liquid or food may be placed into sealed containers and shipped to a central analytical facility for analysis. Where appropriate, samples may be refrigerated or frozen prior to shipping and may be shipped at ambient temperature or in a refrigerated or chilled package.

As discussed below, where a binary readout is obtained (a given marker is present or absent), a binary coding system employing combinations of different markers may be employed. Where the profit margin of counterfeiting is sufficient to justify the expense, it is possible that reverse engineering could be employed to determine what markers have been added to a product. The counterfeiter could then add the same markers to the fake product to circumvent the testing process. To avoid that possibility, the markers utilized to mark a given product could be subject to a rotating system, where the two or more markers added to a given product may vary as a function of date of manufacture, lot number, production facility, a numerical identifier on a product label, or any other factor of choice. This would greatly complicate the task of the counterfeiter, as the number of possible binary codes that could be employed is limited only by the number of possible markers. Further, there is no a priori reason why the number of markers added to the product is limited to two. As the number of markers added to each product increases, the number of possible combinations of markers expands rapidly. Although preferred embodiments utilize combinations of two markers, it is contemplated that other embodiments may utilize 3, 4, 5 or even more markers added to a product.

In alternative embodiments, where more precise analytical methods are employed such as HPLC or mass spectroscopy, the concentration of each marker present in the sample may be determined. In this case, a more sophisticated coding scheme may be utilized. For example, if a first marker is added at concentration “X”, the second marker could be added at a concentration of 0.5X, 1X, 2X, 3X, 4X, etc. In addition to the combinations of markers used, the relative concentrations of the markers in the product may be an independent variable in the coding scheme, resulting in a code with a large number of possible permutations that would be very difficult to circumvent.

To provide additional security for the coding scheme, it is possible to have the markers formulated and prepared in a separate facility and shipped in a sealed container to the packaging plant, so that the personnel responsible for making and packaging the product would not know the coding scheme used for a particular lot or batch. For example, where a bottling operation is responsible for packaging a liquor, the bottler could not merely run off and package an additional batch of product for off-market distribution, since the volume and concentration of marker compounds could be formulated to be sufficient only for the approved amount of product.

Markers

As discussed above, it is contemplated that a wide variety of known compounds could be used as potential markers in the practice of the claimed methods. In certain preferred embodiments, the markers may be vitamins, preferably vitamins that are soluble in liquors, more preferably vitamins that are not routinely added to the liquid or food products that are to be marked. For example, since milk typically contains added vitamin D, it would be inappropriate for milk products to be marked with a combination including vitamin D.

Non-limiting examples of markers that may be of use include vitamin B-1 (thiamine), vitamin B-2 (riboflavin), vitamin B-3 (niacin), vitamin B-5 (pantothenic acid), vitamin B-6 (pyridoxine), vitamin B-12 (cobalamin), vitamin A (beta-carotene), vitamin C (ascorbic acid), vitamin D (cholecalciferol), vitamin E (tocopherol), biotin, avidin, streptavidin, folic acid, choline, inositol, omega 3 fatty acids, boron, calcium, magnesium, chromium, copper, iron, zinc, potassium, selenium, glutamine, tyrosine, cysteine, carnitine, glutathione, lysine, methionine and tryptophan.

Binary Code Label

In certain preferred embodiments, a label containing a code system may be used as a supplemental marking system to distinguish genuine from counterfeit products. For example, such a label could be incorporated into the product packaging and could encode information on what two or more marker substances should be present in the liquid or food product. The label and marker system could be read together to provide a more secure and difficult to circumvent validation system. An exemplary binary code label comprising fluorescent or luminescent spots is disclosed in U.S. patent application Ser. No. 10/425,222, filed Apr. 29, 2003, comprising spots that are read in a binary (plus/minus or zero/one) system.

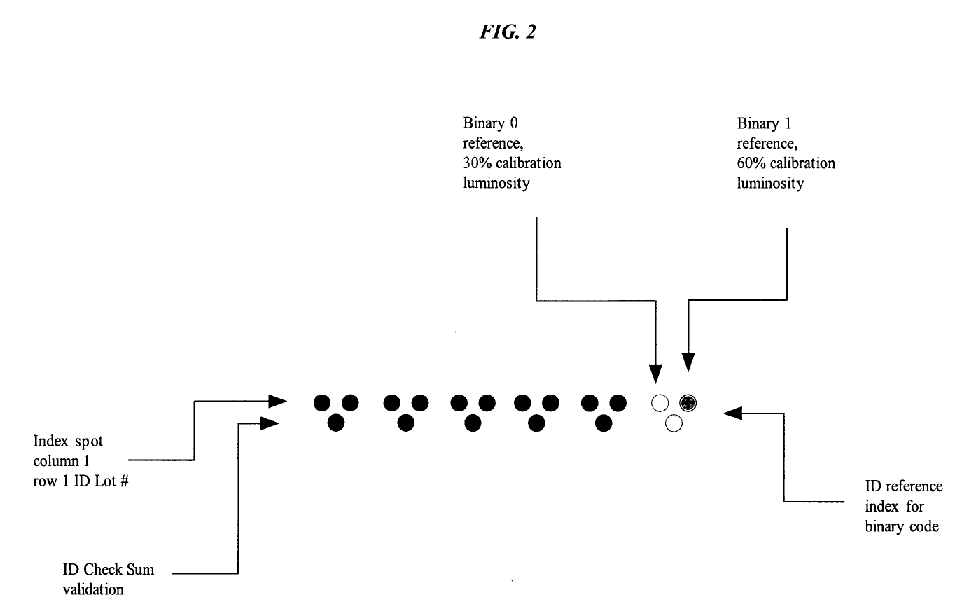

FIG. 2 illustrates an exemplary binary code system to identify, for example, a particular production lot or a date of manufacture. The figure shows a set of two rows of spots, each of which may contain a fluorescent or luminescent tag. Non-limiting examples of fluorescent or luminescent tags of use are disclosed below. In one example, a fluorescent label may be illuminated with a UV light source and the binary code read by a simple hand-held scanner, many examples of which are known in the art.

There are 3 reference spots in the first calibrator position, in this example on the far right side. The reference spots are used to determine if the other spots in the adjacent columns are binary 1 or binary 0. A 4^{th} spot located at the coordinates for row 2 column 2 is a quality assurance check sum on the intended code, for example, for the lot number assigned. The other spots in the two rows are either empty or filled with a fluorescent tag at a concentration and volume that is about three times the level of the binary 1 spot (see below) and are determined to be either 0 or 1 by comparison to the 3 binary reference marks.

Binary 0 Reference Spot

The binary 0 reference spot contains 1/10^{th} of the concentration of the fluorescent tag used in a standard calibration spot. If any of the spots used to set the binary code are measured with a luminosity of lower intensity than the binary 0 reference, they are assigned a value of 0. A spot measured with a greater luminosity than both the binary 0 reference and the binary 1 reference is assigned the value of 1. A spot with a measured luminosity in between the binary 0 and binary 1 reference spots registers as a defective label (or counterfeit label).

Binary 1 Reference Spot

The binary 1 reference spot contains 3/10^{th} of the concentration of the fluorescent tag used in a standard data spot. If any of the spots used to set the binary code are measured with a luminosity of greater intensity than the binary 1 reference, they are assigned a value of 1. A spot of lower luminosity than both the binary 1 reference and the binary 0 reference is assigned the value of 0.

Binary Code and Lot Number Assignments

In an exemplary embodiment, for each production run of a liquid or food product there is a lot production date. The date of production may serve as the Lot number assignment for that production. The date may be printed in a binary code format. The unique pattern for a particular production date may be determined when a request for a lot production run is made.

A software program coding and assigning filled and empty spots may be used so that a user by entering the date for the production run will see the spots that are to be filled and those to be left empty. The program used for the pattern of filled spots in the ID code also generates the assignment for the check-sum validation reference.

Check Sum Validation Spot

A reference spot at row 2 column 2 may be either completely filled or left empty as a validation of the binary code on the label. If the sum of the value 1's in the binary code assigned for a particular date is an even number, then the value 0 (empty spot) may be assigned for the check sum validation spot. If the sum of the value 1's in the binary code assigned for a particular date is an odd number, then the value 1 (filled spot) may be assigned for the check sum validation spot. The check sum validation when read against the binary code reference spots will be either 0 or 1. If the value read fails to match the assigned value for a particular production date, the label is identified as either defective or counterfeit.

### EXAMPLES

### Example 1

### Exemplary Embodiment of a Method for Detecting Counterfeit Liquids and/or Foods

The skilled artisan will realize that many different markers and analytical techniques for detecting markers may be utilized within the claimed methods. The following is provided as a non-limiting example of a potential marker and analytical technique of use.

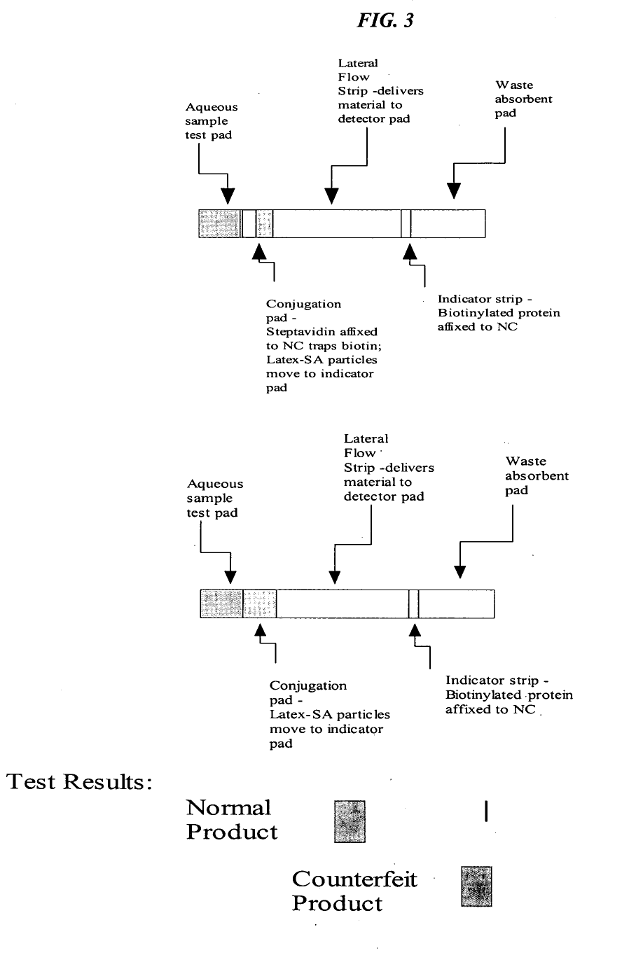

FIG. 3 illustrates a non-limiting example of a marker and analytical detection technique of use in the claimed methods. The exemplary analytical technique utilizes a lateral flow “dip-stick” type of analysis. Material for lateral flow test strips is commercially available, for example from Pall Corporation (East Hills, N.Y.). A typical lateral flow strip comprises a microporous material, such as nitrocellulose, cellulose acetate or a glass fiber membrane. In the exemplary embodiment disclosed in FIG. 3, nitrocellulose (NC) is used for the microporous membrane. NC is typically brittle and fragile as a pure sheet, so it is laminated onto a semi-rigid plastic substrate, such as polyester, styrene or polyvinyl chloride (PVC), often using a pressure-sensitive or heat-sensitive adhesive.

In the illustrative example of FIG. 3, the test strip is divided lengthwise in half. Both halves have an absorbent pad, for example, a paper-based product, glass fibers or polypropylene, to which a sample is applied. In one embodiment, the end of the test strip containing the absorbent pad may be dipped into a liquid to be tested. A waste absorbent pad is present at the other end of the test strip. This serves to create a lateral flow wicking action that moves the sample liquid (and any markers contained in the liquid) across the test strip from the sample test pad to the waste absorbent pad.

The marker substance, in this example, is biotin. In the positive control half of the test strip, there is a conjugation pad located just upstream from the sample test pad. The conjugation pad contains an initial zone of streptavidin or avidin covalently attached to the nitrocellulose. The streptavidin binds to any biotin that is present in the sample. The conjugation pad also contains colored microparticles of latex covalently conjugated to streptavidin (SA) (obtained, for example, from Pall Corp.) The wicking action created by the waste absorbent pad results in lateral fluid flow, carrying the colored latex-SA particles along the test strip. At the indicator strip, biotinylated proteins are covalently attached to the nitrocellulose membrane. Because the colored latex-SA particles are not already saturated with biotin, they bind to the indicator strip and stop moving, resulting in the formation of a colored strip on the top half (positive control) of the strip.

On the bottom half of the test strip, there is no avidin or streptavidin attached to the nitrocellulose in the conjugation pad, only colored latex-SA particles. The mobility of the particles depends upon the presence or absence of the biotin marker in the sample. Where biotin is present (genuine or “normal” product), it saturates the SA attached to the latex particles. When the particles reach the indicator strip containing biotinylated protein, the particles continue to migrate towards the waste absorbent pad, rather than being immobilized on the indicator strip. Thus, the readout from the normal (genuine) product shows a colored line on the positive control half of the test strip, but no colored line on the bottom test portion of the strip.

A counterfeit product does not contain the biotin marker. Thus, the colored latex-SA particles are not saturated with biotin when they arrive at the indicator strip. They therefore bind to the immobilized biotinylated protein affixed to the indicator strip to form a colored line that extends all the way across the test strip. The presence of a continuous colored line extending across the test strip is a positive indicator of a counterfeit product, lacking the biotin marker.

The skilled artisan will realize that although the example of FIG. 3 shows detection of a single marker compound (biotin), the same assay may be adapted to detect two or more marker compounds. The two or more different markers may be detected using a single test strip, for example containing two different indicator strips with different capture molecules to immobilize two different types of conjugated latex particles, or by using two different test strips, each of which can detect the presence or absence of a single marker compound.

The skilled artisan will further realize that although the exemplary embodiment utilizes a biotin-streptavidin binding pair, the assay can be adapted to work with any marker substance for which a binding molecule is available. For example, the lateral flow technique is commonly used with immunochromatography, wherein one or more antibodies are available to bind to and detect the marker substance. Immunochromatographic techniques with lateral flow devices are well known in the art, for example U.S. Pat. No. 6,194,225 (incorporated herein by reference from Col. 7, line 31 through Col. 12, line 40). In general, any marker substance for which one or preferably two antibodies are available may be used in a lateral flow technique. As discussed above and as is well known in the art, antibodies are commercially available for many of the proposed exemplary marker compounds listed above.

What is claimed is:

1. A method for detecting a counterfeit liquid or food comprising:
a) adding two or more markers to a liquid or food product;
b) distributing and/or selling the product;
c) obtaining a sample purported to be the liquid or food product; and
d) analyzing the sample for the presence of the two or more markers;
wherein the absence of any of the two or more markers from the sample indicates that the product is counterfeit.

2. The method of claim 1, wherein the two or more markers are vitamins.

3. The method of claim 1, wherein the two or more markers are selected from the group consisting of vitamin B-1 (thiamine), vitamin B-2 (riboflavin), vitamin B-3 (niacin), vitamin B-5 (pantothenic acid), vitamin B-6 (pyridoxine), vitamin B-12 (cobalamin), vitamin A (beta-carotene), vitamin C (ascorbic acid), vitamin D (cholecalciferol), vitamin E (tocopherol), biotin, avidin, streptavidin, folic acid, choline, inositol, omega 3 fatty acids, boron, calcium, magnesium, chromium, copper, iron, zinc, potassium, selenium, glutamine, tyrosine, cysteine, carnitine, glutathione, lysine, methionine and tryptophan.

4. The method of claim 1, wherein the product is a liquor.

5. The method of claim 4, wherein the liquor is selected from the group consisting of beer, wine, champagne, vermouth, vodka, scotch, whiskey, bourbon, brandy, cognac, gin, rum, tequila, schnapps, absinthe, akvavit, amaretto, amarula, arak, cassis, compari, Contreau, curacao, Drambuie, Galliano, Grand Marnier, Jagermeister, kahlua, kirsch, Midori, ouzo, pernod, sambuca, triple sec and sake.
